(19) United States
(12) Patent Application Publication
(10) Pub. No.: US 2007/0055167 A1
(43) Pub. Date: Mar. 8, 2007

(54) System and method for analysis of cardiac arrhythmia using timing and variability of relationships between elctrogram features

(76) Inventor: John R. Bullinga, La Jolla, CA (US)

Correspondence Address: FAY KAPLUN & MARCIN, LLP, 15O BROADWAY, SUITE 702, NEW YORK, NY, 10038, US

(21) Appl. No.: 11/219,586

(22) Filed: Sep. 2, 2005

(51) Int. Cl. A61B 5/04

(52) U.S. Cl. 600/509

(57) ABSTRACT

Described is a system and method for recording and receiving a plurality of electrograms, each electrogram corresponding to a region of a heart, wherein the electrograms are recorded substantially simultaneously. A plurality of fiducial markers are identified on a selected electrogram, wherein each fiducial marker marks a feature of interest in the first electrogram. Multiple segments of each of the plurality of electrograms are aligned with each segment temporally corresponding to each of the fiducial markers on the selected electrogram. The aligned segments of each of the plurality of electrograms are averaged resulting in an average electrogram for each of the plurality of electrograms. Features of interest are identified in each of the plurality of average electrograms and relationships are determined between features of interest in each of the plurality of average electrograms.

### BACKGROUND INFORMATION

Cardiac arrhythmias are a common problem in the United States and these arrhythmias may result in significant morbidity. Electrophysiologic study of patients with cardiac arrhythmias is commonly performed to identify the mechanism of the arrhythmia. Once the region of cardiac tissue that is critically involved in the mechanism of the arrhythmia is identified, therapies such as ablation may be delivered to disrupt the mechanism of the arrhythmia. These therapies may be curative for the arrhythmia. Commonly, the identification of critical areas of cardiac tissue that are involved in the mechanism of the arrhythmia is difficult. Methods for identification of critical areas mechanistically involved in arrhythmias may facilitate delivery of therapies that cure arrhythmias.

### SUMMARY OF THE INVENTION

A method for receiving a plurality of electrograms, each electrogram corresponding to a region of a heart, wherein the electrograms are recorded substantially simultaneously. A plurality of fiducial markers are identified on a selected electrogram, wherein each fiducial marker marks a feature of interest in the first electrogram. Multiple segments of each of the plurality of electrograms are aligned with each segment temporally corresponding to each of the fiducial markers on the selected electrogram. The aligned segments of each of the plurality of electrograms are averaged resulting in an average electrogram for each of the plurality of electrograms. Features of interest are identified in each of the plurality of average electrograms and relationships are determined between features of interest in each of the plurality of average electrograms.

A method for identifying a first plurality of electrogram features in a first electrogram, identifying a second plurality of electrogram features in a second electrogram and determining a relationship between the first plurality of features and the second plurality of features.

A method for receiving a plurality of electrograms, each electrogram corresponding to a region of a heart, wherein the electrograms are recorded substantially simultaneously, identifying a plurality of fiducial markers in each electrogram, wherein each fiducial marker marks a feature of interest in the electrogram and comparing the fiducial markers in the electrograms to determine relationships between the features of interest.

A signal processing device comprising a processor and a memory storing a set of instructions. The instructions being operable to identify a first plurality of electrogram features in a first electrogram, identify a second plurality of electrogram features in a second electrogram and determine a relationship between the first plurality of features and the second plurality of features.

A system including a plurality of catheters recording a plurality of electrograms, each electrogram corresponding to a region of a heart, wherein the electrograms are recorded substantially simultaneously. The system further including a signal processing device to receive the plurality of electrograms, identify a plurality of fiducial markers on a selected electrogram, wherein each fiducial marker marks a feature of interest in the first electrogram, align multiple segments of each of the plurality of electrograms, each segment temporally corresponding to each of the fiducial markers on the selected electrogram, average the aligned segments of each of the plurality of electrograms resulting in an average electrogram for each of the plurality of electrograms, identify a feature of interest in each of the plurality of average electrograms and determine relationships between features of interest in each of the plurality of average electrograms.

A signal processing device comprising a processor and a memory storing a set of instructions. The instructions being operable to receive a plurality of electrograms, each electrogram corresponding to a region of a heart, wherein the electrograms are recorded substantially simultaneously, identify a plurality of fiducial markers in each electrogram, wherein each fiducial marker marks a feature of interest in the electrogram and compare the fiducial markers in the electrograms to determine relationships between the features of interest.

### BRIEF DESCRIPTION OF DRAWINGS

FIG. 1 shows an exemplary illustration of a reentrant arrhythmia.

FIG. 2 shows an exemplary general diagnostic method according to the present invention to determine relationships between electrograms and select regions for therapeutic intervention.

FIG. 3A shows an exemplary technique to signal process multiple electrograms according to the present invention.

FIG. 3B shows an exemplary averaging technique to identify the least variable electrogram features between two electrograms according to the present invention.

FIG. 3C shows an exemplary averaging technique to identify the least variable electrogram features between multiple electrograms according to the present invention.

FIG. 4 shows an exemplary pattern based technique to analyze the timing and variations in relationships between multiple electrograms according to the present invention.

FIG. 5 shows an exemplary illustration of a reentrant arrhythmia with multiple recording regions according to the present invention.

FIG. 6A shows an exemplary illustration of reentrant arrhythmia with the roving catheter electrogram preceding cycle length changes in the reference catheter according to the present invention.

FIG. 6B shows an exemplary illustration of an averaging technique with the roving catheter electrogram preceding cycle length changes in the reference catheter according to the present invention.

FIG. 7A shows an exemplary illustration of reentrant arrhythmia with the roving catheter electrogram following cycle length changes in the reference catheter according to the present invention.

FIG. 7B shows an exemplary illustration of an averaging technique with the roving catheter electrogram following cycle length changes in the reference catheter according to the present invention.

FIG. 8 shows an exemplary illustration of a focal arrhythmia with multiple recording regions according to the present invention.

FIG. 9A shows an exemplary illustration of focal arrhythmia with the roving catheter electrogram preceding cycle length changes in the reference catheter according to the present invention.

FIG. 9B shows an exemplary illustration of an averaging technique with the roving catheter electrogram preceding cycle length changes in the reference catheter according to the present invention.

FIG. 10 shows an exemplary pattern based technique to analyze the timing and variations in relationships between multiple electrograms according to the present invention.

FIG. 11 shows an exemplary table of statistics to determine the temporal relationship and temporal variation in timing between electrogram features according to the present invention.

### DETAILED DESCRIPTION

The present invention may be further understood with reference to the following description and the appended drawings, wherein like elements are provided with the same reference numerals. The present invention includes a diagnostic method that detects the timing and variability between electrogram features in multiple electrograms obtained during an arrhythmia. Information of the relationships between electrogram features may then be incorporated into a three-dimensional mapping system to identify temporal, spatial and variability in relationships between electrogram features. The present invention further includes methods to identify regions that are critically involved in generating the cycle length (CL) variations. These regions that produce the CL variations may be mechanistically involved in perpetuating the arrhythmia and may be targeted for further therapies to cure the arrhythmia.

Reentrant arrhythmias involve a mechanism of an endless loop whereby activation proceeds in a loop around a fixed or functional region of block. The time between electrical activations of a region of the heart is the cycle length of the arrhythmia. For a reentrant arrhythmia, the cycle length is the time for electrical activation to complete one loop around the circuit. In general, a large portion of the loop conducts rapidly with little variation of conduction velocity. A small portion of the loop conducts slowly with larger variation of conduction velocity. In general, the area of slow conduction is a region of diseased tissue. Identification of this region of diseased tissue yields a target for additional therapies to cure the arrhythmia.

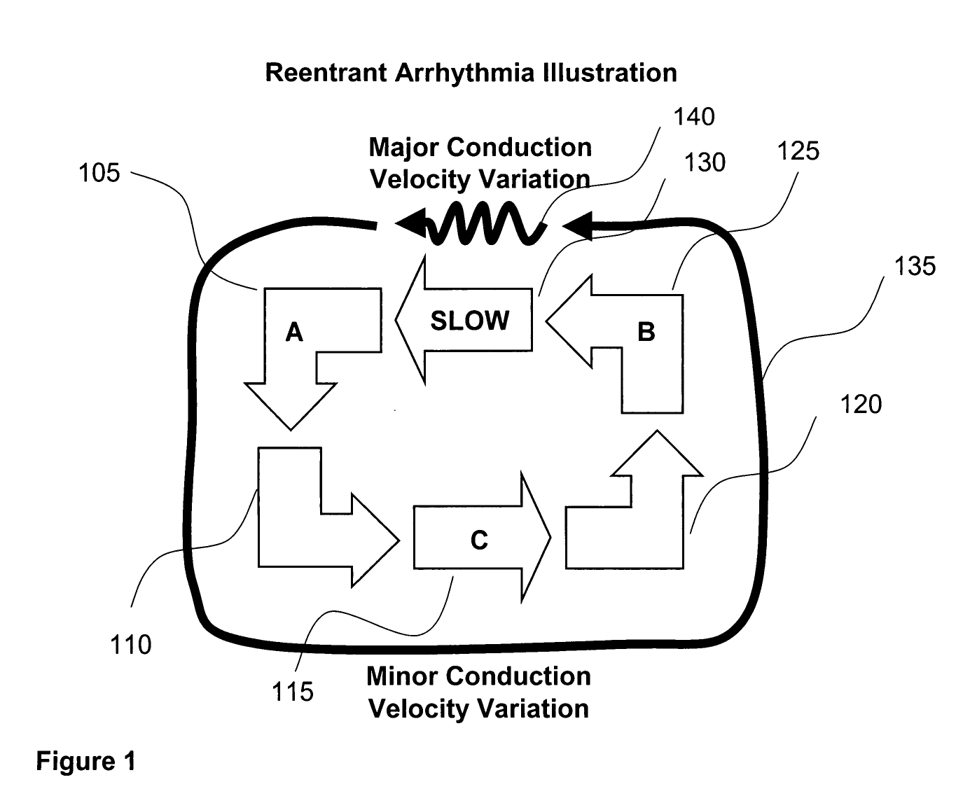

FIG. 1 shows an exemplary illustration of a reentrant arrhythmia. The arrhythmic loop is represented by a series of blocks 105-130. As is shown by the arrow block 130 labeled as “slow”, this region has slow conduction velocity. Regions with slow conduction have more variation in conduction velocity than regions with rapid conduction. This is further illustrated by the conduction velocity waves 135 and 140. Consequently, regions with slow conduction contribute more to cycle length variation than regions with rapid conduction. The other arrow blocks 105-125 have fast conduction and are a minor source of cycle length variation due to having less variation of conduction velocity.

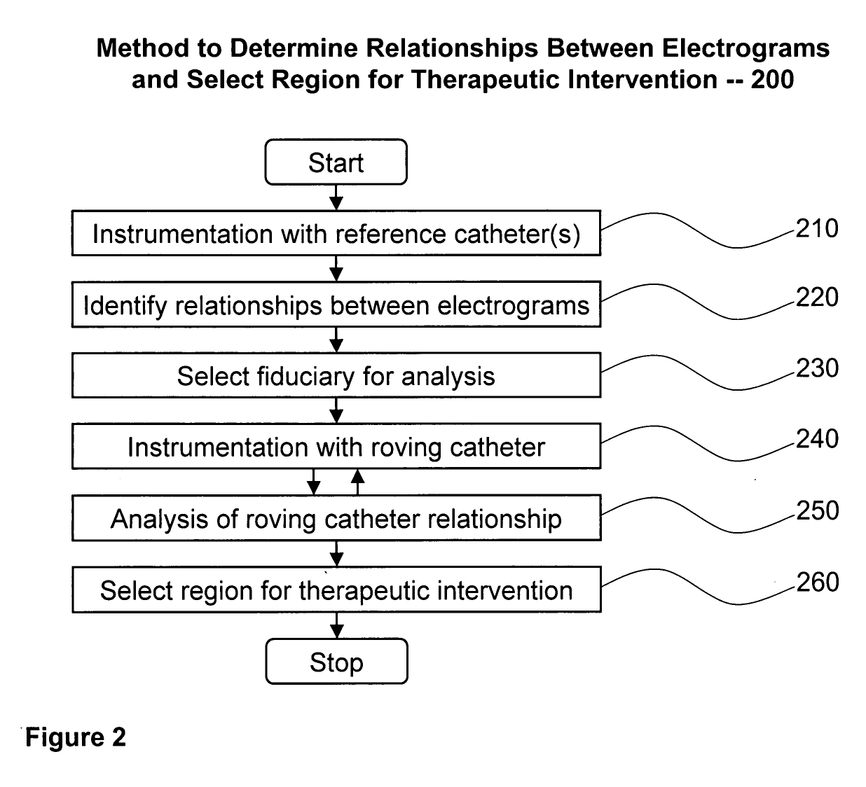

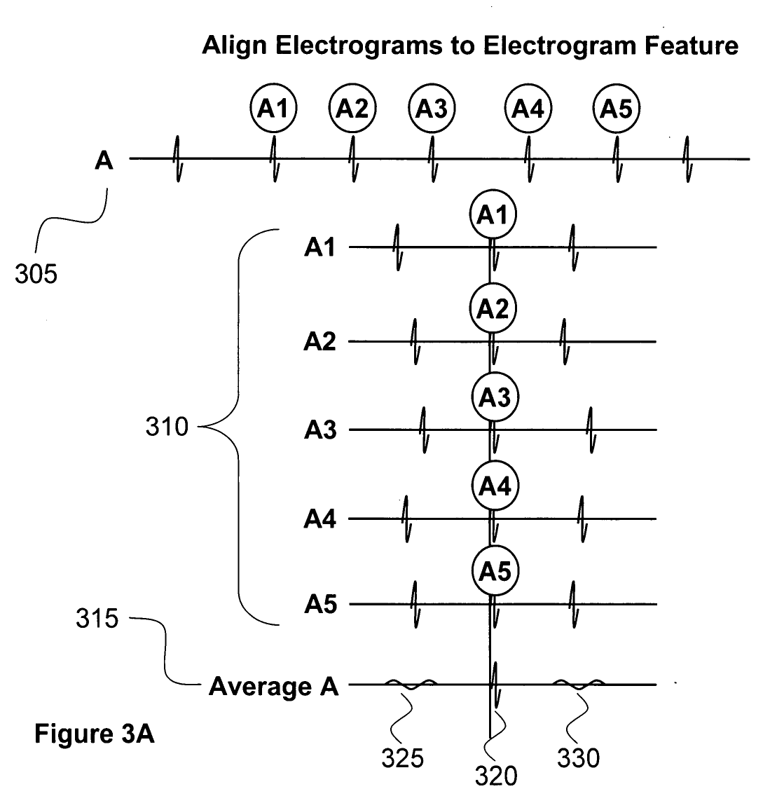

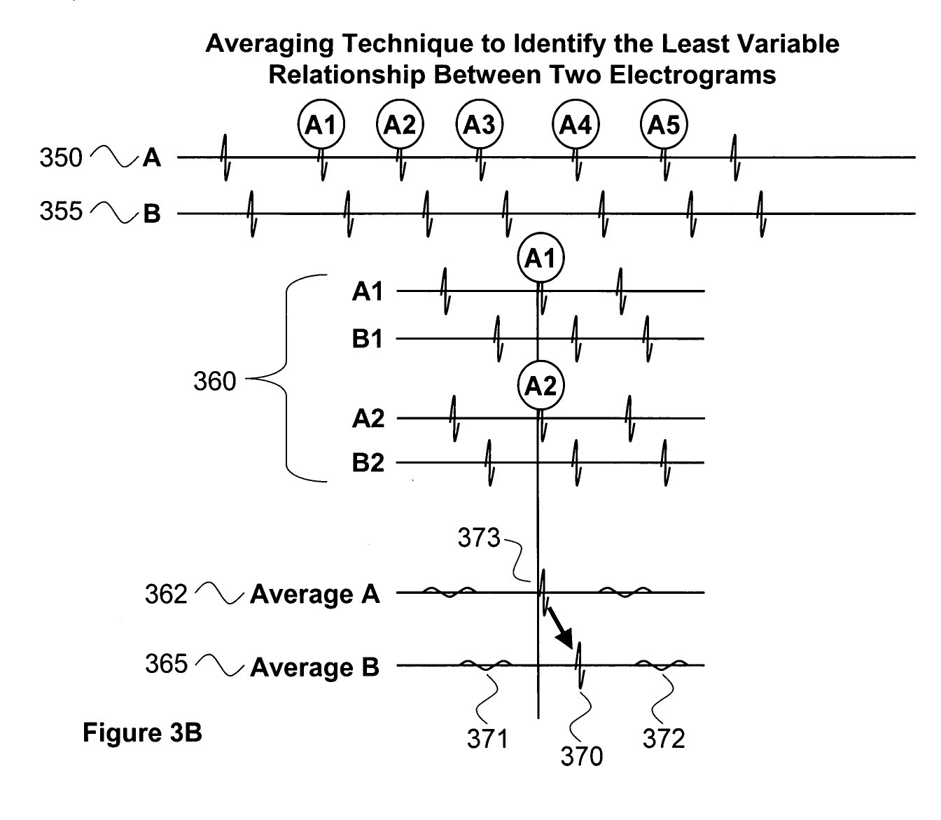

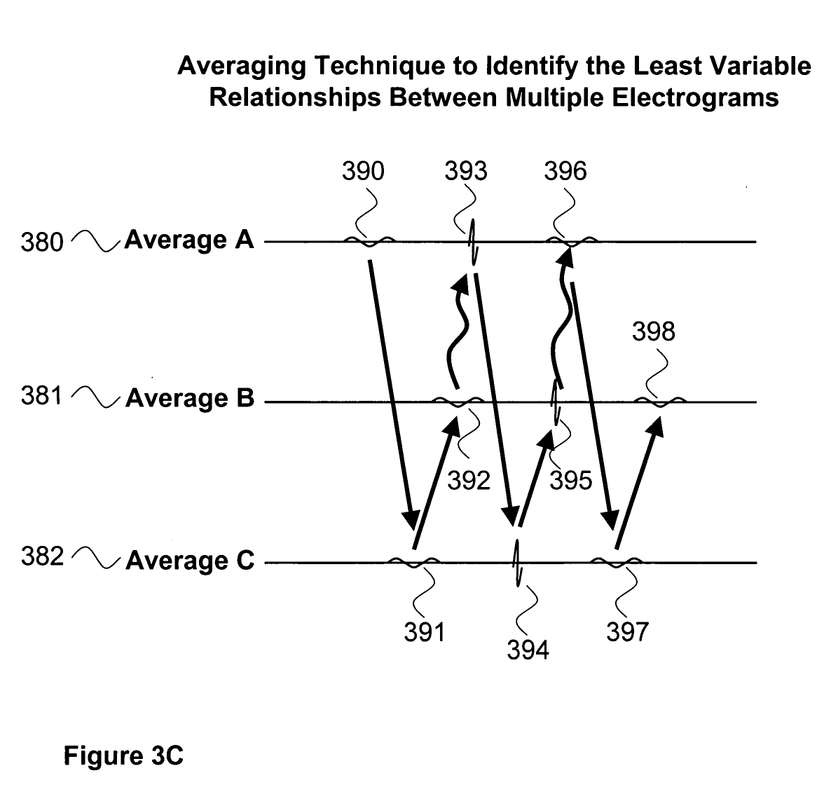

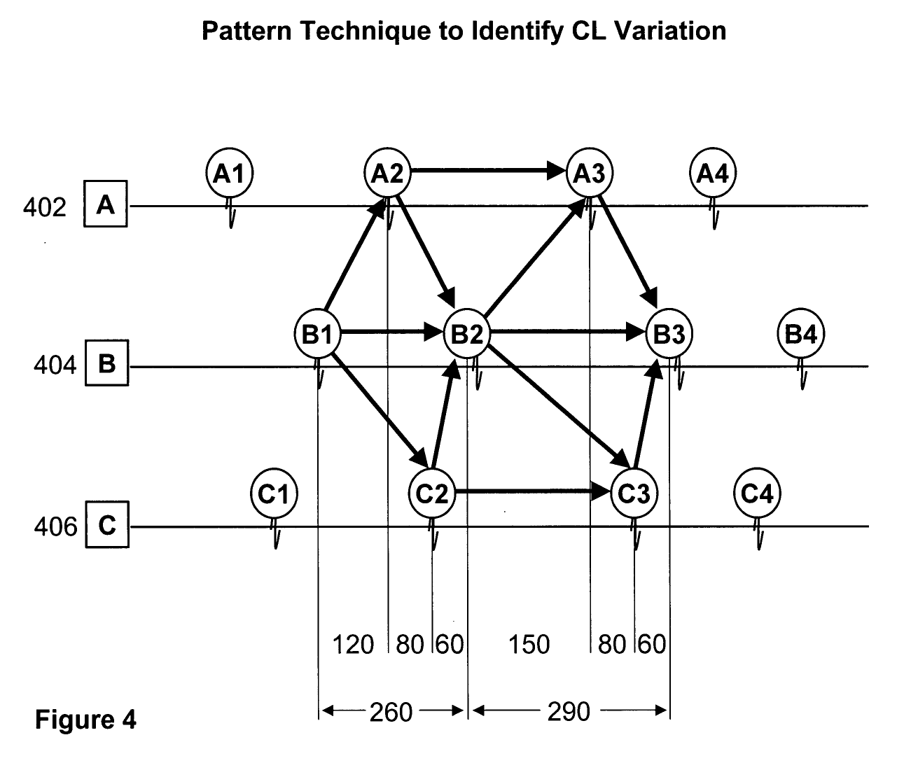

FIG. 2 shows an exemplary general diagnostic method according to the present invention to determine relationships between electrograms and select regions for therapeutic intervention. This process starts with step 210 wherein the heart is instrumented with one or more reference catheters. These catheters are then used to provide electrogram signals to a recording system. Each of the electrogram signals is recorded simultaneously. Software within the recording system or on a separate computer may then analyze those electrograms. In step 220, methods described below with reference to FIGS. 3 and 4 are used to determine the relationships between features of each of the electrograms. In step 230, methods described below are used to select an electrogram feature as a fiduciary for further analysis of the arrhythmia. This fiduciary may represent an electrogram feature in a specific electrogram or a composite of electrogram features from multiple electrograms.

In step 240, the heart is instrumented with an additional catheter, called a “roving” catheter. This catheter may be re-positioned in multiple additional regions for recording of electrograms, specifically including regions where the reference catheters are not positioned. In step 250, the electrogram from the roving catheter is analyzed relative to the electrograms from the reference catheters. This step includes continued analysis of the relationships between reference catheters electrograms and analysis of the relationship of the roving catheter electrogram to the reference electrograms. This step may further include recording the relationships on a three-dimensional mapping system as discussed below.

In step 260, a region is selected for therapeutic intervention. The region is selected based on being an area that is critically involved in the mechanism of the arrhythmia. This region may be the region with the electrogram that has the earliest activity with least variation relative to other electrogram features on the reference catheter electrograms or other roving catheter electrograms. The region may also be selected as the region between the regions with earliest and latest activity that have the least temporal variation among the electrogram features. Alternatively this region may be selected for other standard electrophysiologic criteria with adjunctive information from the relationships between electrogram features.

The following is a description of an exemplary method of identifying relationships between electrograms as described with reference to step 220 of FIG. 2. FIG. 3A shows an exemplary technique to align electrograms to an electrogram feature according to the present invention. The electrogram ‘A’ labeled 305 may be the electrogram collected by a reference catheter placed in a location corresponding to the block 105 (labeled ‘A’) of FIG. 1. In this illustration of electrogram ‘A’ labeled 305, the features of interest are the short duration high frequency events labeled ‘A1’, ‘A2’, ‘A3’, ‘A4’, and ‘A5’. During a typical arrhythmic event, this series would continue for a pre-specified duration or on a continuous buffered basis with a pre-specified size of the buffer. A signal processing technique may be used to automatically identify each of the short duration high frequency events A1-A5. Each of the short duration high frequency events will serve as a fiducial point for analysis. The available techniques to identify these events include but are not limited to maximum rate of voltage change, the maximum or minimum voltage in a specified region, or the maximum correlation to a template. Data matrix 310 of FIG. 3A is then created with electrogram data buffered relative to the fiducial point with data including before and after the fiducial point. Each of the data labels are aligned to the fiducial within the data matrix as is illustrated in FIG. 3A.

Multiple methods may be applied to process the data matrix of electrograms. A direct average of the voltage may be applied. Alternatively, the absolute value of the voltage may be averaged. Other methods such as the standard deviation may be measured for each point in time along the electrogram. A combination of these or additional measures may be used to select the best method to identify electrogram feature of interest. In FIG. 3A, the data matrix is processed with a direct average to produce the electrogram ‘Average A’ as illustrated and labeled as 315. At the fiducial point, the morphology of the short duration high frequency electrogram is preserved and labeled as 320. One cycle length before and after the fiducial point, the short duration high frequency electrogram is lengthened in duration and reduced in amplitude as labeled as 325 and 330 due to averaging with the variation of the cycle length of the arrhythmia.

FIG. 3B shows an exemplary averaging technique to identify the least variable electrogram features between two electrograms according to the present invention. Similar to FIG. 3A, electrogram ‘A’ labeled 350 has sequentially labeled short duration high frequency components identified. The electrogram ‘A’ labeled 350 may be the electrogram collected by a reference catheter placed in a location corresponding to the block 105 (labeled ‘A’) of FIG. 1 and the electrogram ‘B’ labeled 355 may be the electrogram collected by a reference catheter placed in a location corresponding to the block 125 (labeled ‘B’) of FIG. 1. Also similar to FIG. 3A, data matrix 360 is created with the electrogram data buffered relative to the fiducial point with data before and after the fiducial point. Each of the data labels are aligned within the data matrix as illustrated in FIG. 3B labeled as 360. Note that electrograms ‘A’ 350 and ‘B’ 355 were obtained simultaneously. Next, electrogram ‘B’ labeled 355 is segmented using the same temporal fiducial points from electrogram ‘A’ and these electrogram segments are placed in the data matrix 360. The result is a data matrix 360 of simultaneous electrogram segments that are aligned to sequential fiducial points on electrogram ‘A’ 350. The data matrix 360 in FIG. 3B only depicts 2 electrogram segments for each electrogram for simplicity of illustration. For purposes of this invention, additional electrogram segments may be used. For example, any number of the blocks 105-130 of FIG. 1 may have a reference catheter placed in a corresponding location to record electrogram data.

Again referring to FIG. 3B, a signal processing technique is applied to the data matrix 360 as discussed above. Direct averaging of electrograms is illustrated in this example, however other signal processing techniques may be applied. Electrogram segments from electrogram ‘A’ 350 are averaged and electrogram segments from electrogram ‘B’ 355 are averaged resulting in two separate average electrogram segments labeled 362 and 365. Again, each of these electrogram segments from both electrogram ‘A’ and ‘B’ were aligned to sequential fiducial points on electrogram ‘A’. The resultant averaged electrogram 362 of FIG. 3B has the same characteristics as electrogram 315 of FIG. 3A as discussed above. The resultant averaged electrogram 365 on FIG. 3B depicts a short duration high frequency electrogram feature labeled 370. Electrogram 365 also depicts that one cycle length before and after the electrogram feature 370, the short duration high frequency electrogram features are lengthened in duration and reduced in amplitude as labeled as 371 and 372. The reduction in magnitude is due to averaging when there is variation of the cycle length of the arrhythmia.

The relationship between electrogram features of average electrogram ‘A’ 362 and average electrogram ‘B’ 365 may now be identified in FIG. 3B. The fidicual marker of average electrogram ‘A’ 362 is electrogram feature 373. In average electrogram ‘B’ 365, there is a short duration high frequency electrogram feature 370 and blunted electrogram features 371 and 372. This indicates that there is little variation in conduction time between the fiducial (feature 373) and the short duration high frequency electrogram feature 370. Additionally, this also indicates that there is greater variation in conduction time between fiducial (feature 373) and the blunted electrogram features 371 and 372. In this example, the region associated with the fiducial (feature 373) is electrically activated before the region associated with the short duration high frequency electrogram feature 370 and there is little conduction velocity variation between the two regions. Also, if this is a reentrant arrhythmia, then there is conduction velocity variation resulting in cycle length variation after activation of the region associated with feature 370 and before returning to the region associated with the fiducial (feature 373).

The relationship between electrogram features may be identified in multiple electrograms that are acquired simultaneously. Again referring to FIG. 1 as an illustration of a reentrant arrhythmia with regions ‘A’, ‘B’ and ‘C’ labeled as 105, 125 and 115, respectively. Electrograms acquired simultaneously from each of these regions are averaged with the signal processing technique discussed above with reference to FIG. 3B and illustrated in FIG. 3C labeled as electrograms 380, 381 and 382. As illustrated in FIG. 1, there is rapid conduction with little conduction velocity variation from blocks ‘A’ 105 to ‘C’ 115 and blocks ‘C’ 115 to ‘B’ 125. The result is that when average electrogram ‘A’ 380 is used as the fiduciary (feature 393), there is preservation of sharp electrogram features in the regions that follow with rapid conduction. The preservation of the signal features is indicated by the sharp electrogram features on average electrogram C 382 (feature 394) and on average electrogram B 381 (feature 395). There are dull electrogram features in regions that require passage through the slow zone of conduction (features 390, 391, 392, 396, 397 and 398). For example, there is activation of region ‘B’ 125 then passage through the slow zone of conduction followed by activation of region ‘A’ 105. Hence, when feature 393 is used as the fiduciary, then electrogram feature 392 is dull. Also, feature 393 is the earliest sharp electrogram feature implying that this feature is after the slow zone of conduction 130 as is illustrated in FIG. 1. Feature 395 is the latest sharp electrogram feature. This implies that the region associated with feature 395 is the region that is last activated in a rapid fashion following activation of the region associated with the fiducial (feature 393).

The description of electrogram analysis with reference to FIG. 1 uses a set of blocks illustrated in two-dimensional space. The heart is a moving three-dimensional structure. Three-dimensional mapping systems are used to associate electrograms with positions in three-dimensional space. Electrogram features may be then mapped in three-dimensional space and onto a model of the heart. The relative or absolute distance between catheter positions where electrograms are collected may be determined with a three-dimensional mapping system. The timing and variability between the electrogram features may be determined using the methods described with reference to FIG. 1. The timing and variability between electrogram features may be plotted in three-dimensional space enhancing the three-dimensional analysis of an arrhythmia.

The following is an alternative exemplary embodiment of a method of identifying relationships between electrograms as described with reference to step 220 of FIG. 2. FIG. 4 shows an exemplary ‘pattern based’ technique to analyze variations in relationships between multiple electrograms. In contrast to the method discussed with reference to FIGS. 3A, 3B and 3C that use signal processing methods such as averaging to determine relationships between electrogram features, the method discussed with reference to FIG. 4 uses a set of fiducial markers to label electrogram features of interest and then determine variations in conduction times between those markers to determine the relationships between those electrogram features.

In reference to FIG. 4, there are three exemplary electrograms, ‘A’ 402, ‘B’ 404 and ‘C’ 406. This technique may be applied to any number of electrograms. Sequential fiducial points A1-A4, B1-B4 and C1-C4 are identified to mark electrogram features that represent specific regional events, such as conduction past the catheter electrodes or regional depolarization. The method then seeks to identify the relationships between each of those fiducial points. In this example, analysis of point ‘B2’ will be performed. First note that the conduction time B1-B2 is 260 ms and the conduction time B2-B3 is 290 ms. These represent the cycle lengths of the arrhythmia and are meant to be illustrative. Smaller cycle length variations may be present in an arrhythmia, however even these smaller cycle length variations may be identified by analyzing a large number of points using the technique being described. The next goal is to identify the relationship between electrogram A 402 and electrogram B 404. The conduction times between the electrogram features are B1-A2 120 ms, A2-B2 140 ms, B2-A3 150 ms and A3-B3 140 ms. The conduction times A2-B2 and A3-B3 are equal implying that A precedes B and that these regions are connected by areas with minor conduction velocity variation. The conduction times between B1-A2 and B2-A3 have large variations that equals the cycle length variation. This implies that there may be a region of marked conduction velocity variation between the two regions. If three-dimensional mapping information is incorporated to identify the spatial relationship between these regions, then a region of slow conduction may be further established.

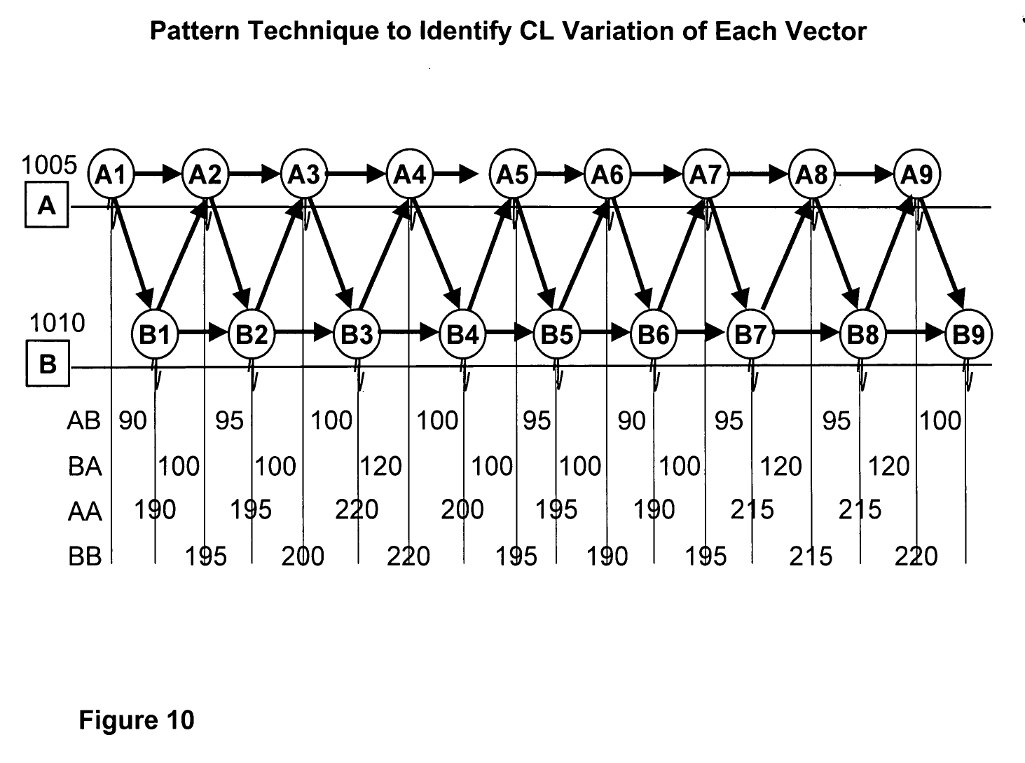

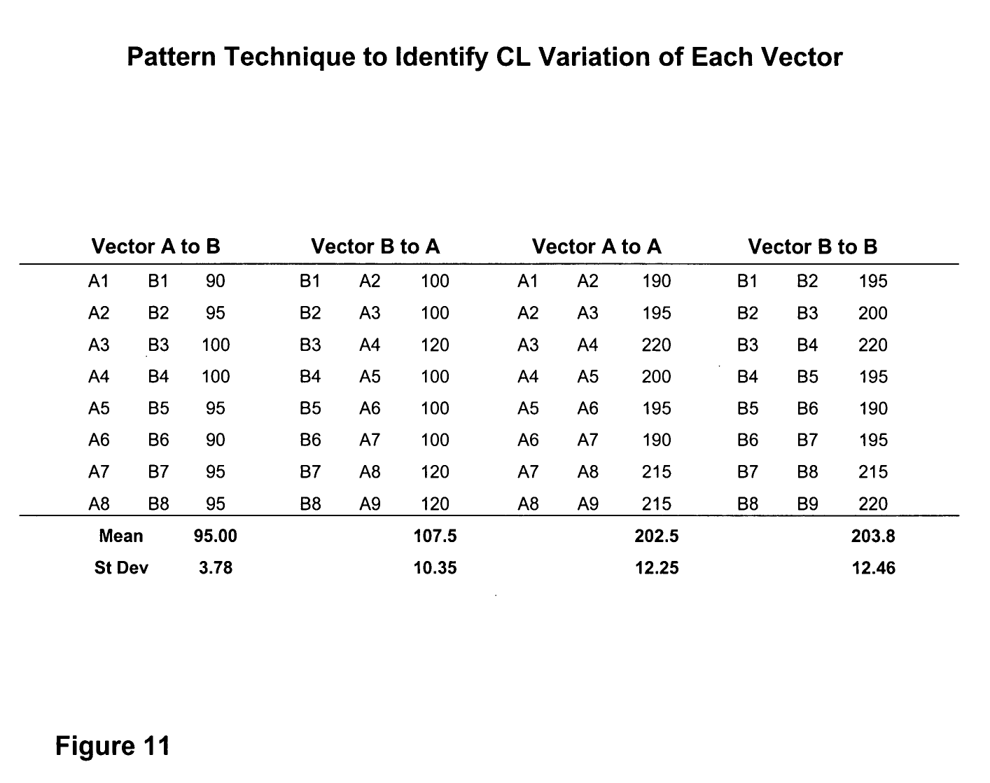

Overall, this technique may be applied to multiple simultaneous electrograms and many fiducial points along each electrogram. Statistics for the cycle length variations and the conduction times between each of the regions may be determined. FIGS. 10 and 11 are illustrations of using statistics to determine the relationship between two electrogram features. Similar to the methods described above with reference to FIG. 4, sequential electrogram features A1-A9 and B1-B9 are marked on electrogram A 1005 and B 1010 (FIG. 10). The conduction times between electrogram features are tabulated and statistics are determined (FIG. 11). In this example, there is little temporal variation from electrogram feature A to B (standard deviation=3.78 ms) and greater temporal variation from electrogram feature B to A (standard deviation=10.35 ms). This implies that there is a zone of variable conduction velocity, which likely includes slow conduction, along the route of electrical activation from B to A. Overall, this technique will allow identification of small variations and relationships between electrograms that are not visually perceptible or identifiable with standard techniques. Similar to the averaging technique discussed with reference to FIGS. 3A, 3B and 3C, regions that are ‘early’ and ‘late’ may be identified.

Those of skill in the art will understand that the preceding averaging technique and pattern technique are provided as examples of methods of identifying cycle length and conduction time relationships between electrogram features that may be used for the purposes of the diagnostic method according to the present invention. However, the present invention may include any number of methods to identify relationships between electrogram features that meet the criteria described above for the diagnostic method.

The next step, as indicated in FIG. 2 block 230 is to select fiducial point(s) or marker(s) for use with further analysis. This fiducial as discussed above may represent an electrogram feature in a specific electrogram or a composite of electrogram features from multiple electrograms. In step 240, the heart is instrumented with an additional catheter, called a roving catheter. This catheter may be positioned in multiple additional regions for recording of electrograms, specifically including regions where the reference catheters are not positioned and where regions are suspected as being critical to the arrhythmia. Analysis of electrograms obtained from the roving catheter will be performed in a similar manner as discussed above to determine the relationship between the roving catheter electrogram and the reference catheter electrograms. When this is performed with a three-dimensional mapping system, information of the relationships found between the roving catheter and the reference catheters may be analyzed in three-dimensional space. When performed multiple times, relationships between multiple positions of the roving catheter may be determined based on relationships determined relative to the reference catheter.

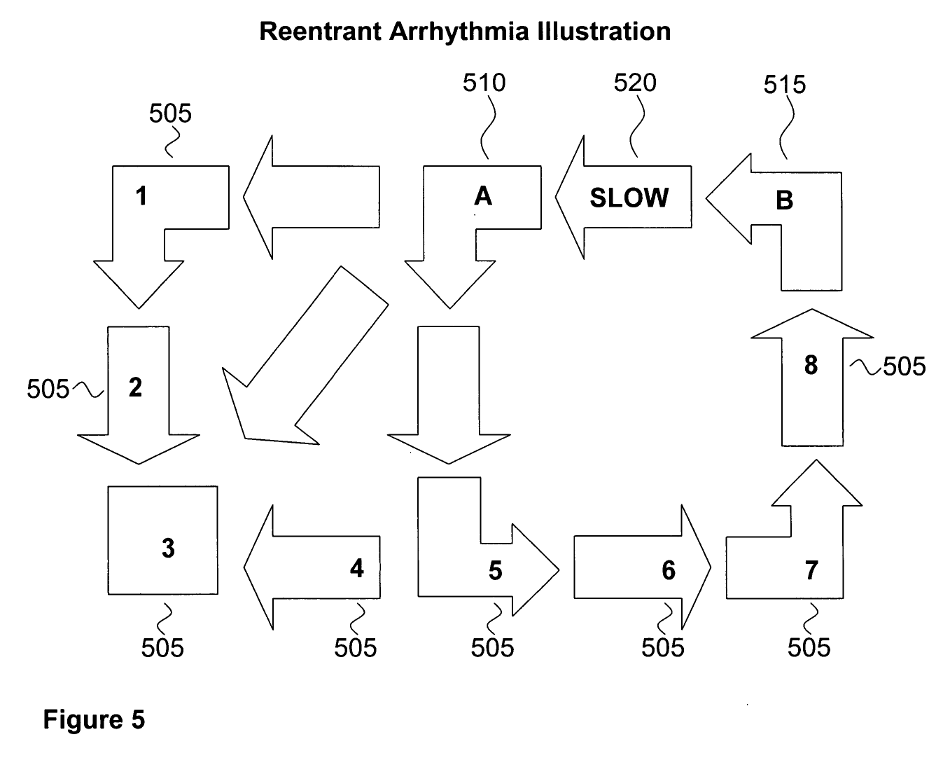

The next step 250 in reference to FIG. 2 is to analyze the roving catheter relative to the reference catheter. FIGS. 5, 6A, 6B, 7A and 7B will be used to explain potential relationships between the roving catheter and the reference catheter for illustrative purposes. Repositioning the roving catheter, step 240 of FIG. 2, and analysis of the roving catheter electrogram relative to the reference catheter, step 250 of FIG. 2, are performed repetitively until adequate information of the arrhythmia is obtained and a therapeutic intervention may be performed, step 260 of FIG. 2.

FIG. 5 shows an exemplary illustration of a reentrant arrhythmia with multiple recording regions according to the present invention. Regions labeled 505 each have a number that represents a reference recording electrogram. Similar to FIG. 1, there are regions A, B and a slow region labeled as 510, 515 and 520, respectively. These regions may be reached with the roving catheter.

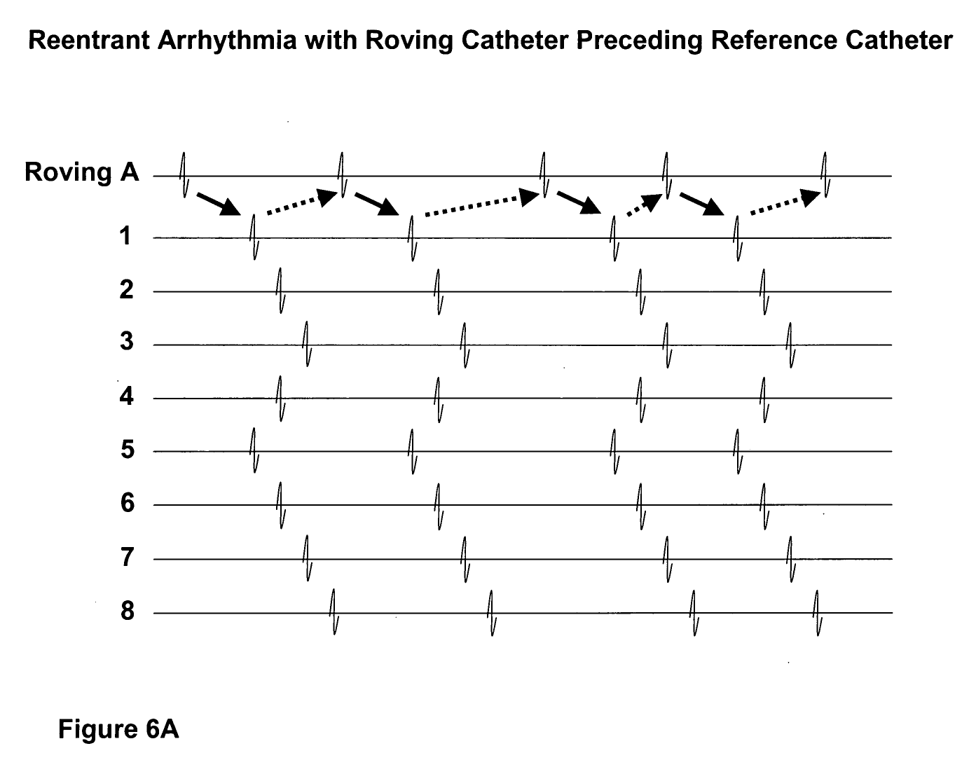

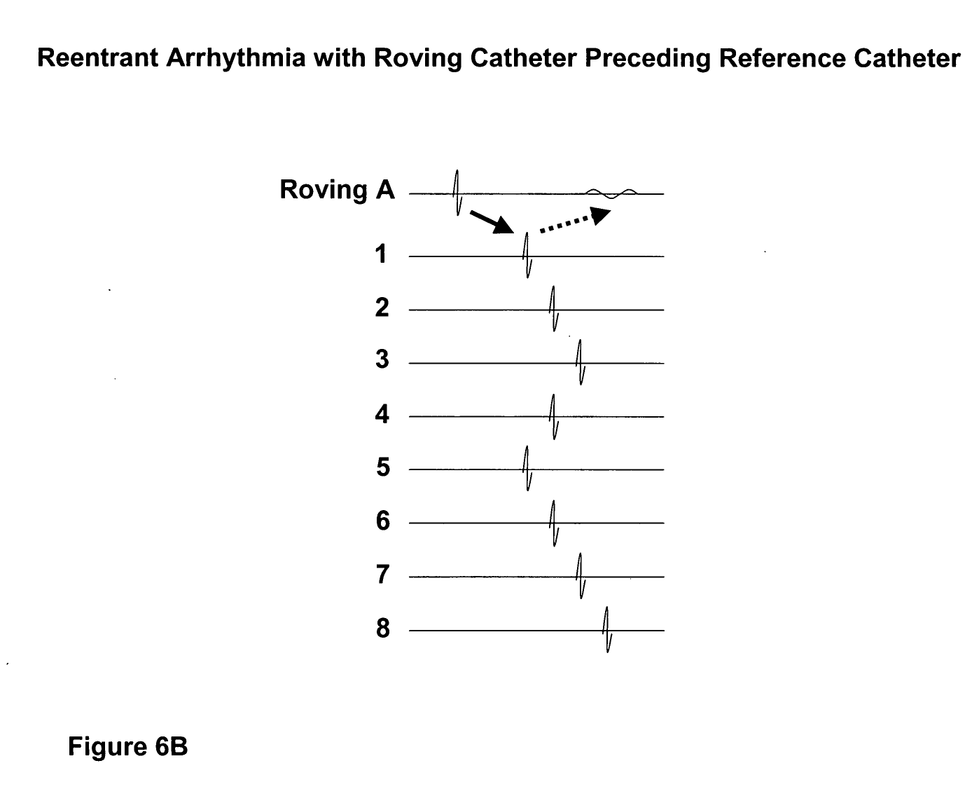

FIG. 6A shows an exemplary illustration of reentrant arrhythmia with the roving catheter in region ‘A’ of FIG. 5 (label 510), i.e, electrogram Roving A is recorded by the roving catheter at the region 510 and electrograms 1-8 are recorded at regions 505 (1-8) by reference catheters. Analysis of this electrogram will show that the electrogram feature corresponding to an activation event precedes cycle length changes in the reference catheter according to the present invention. Using the averaging technique discussed above, FIG. 6B shows an exemplary illustration with the roving catheter electrogram preceding cycle length changes in the reference catheter according to the present invention. Alternatively, the pattern-based technique discussed above could also have been used to determine the relationship between roving catheter and reference catheter features.

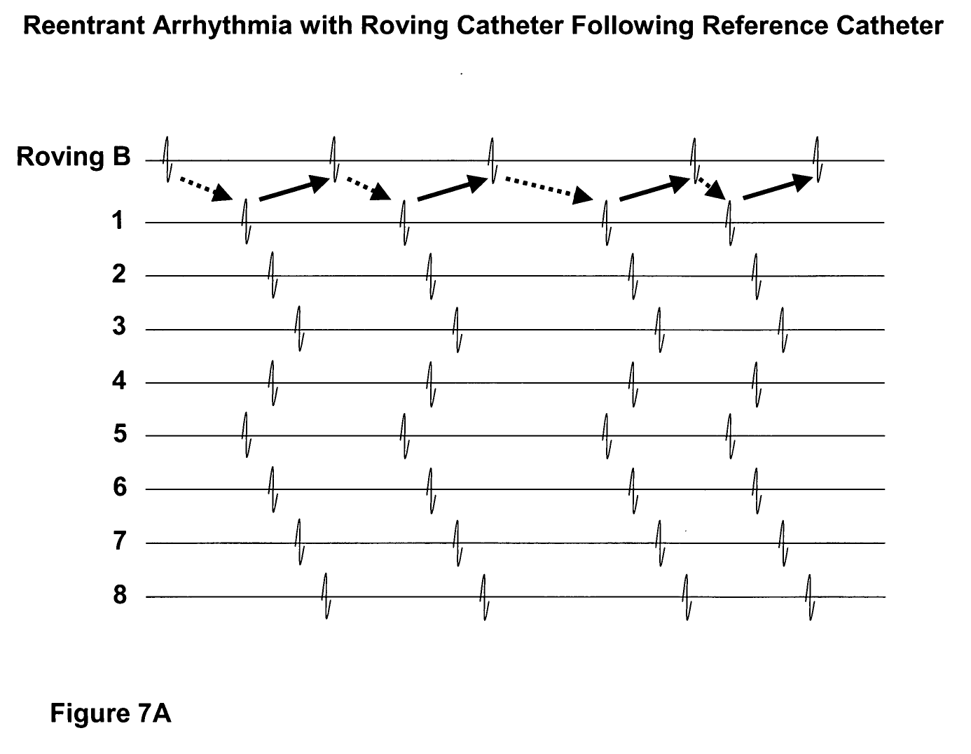

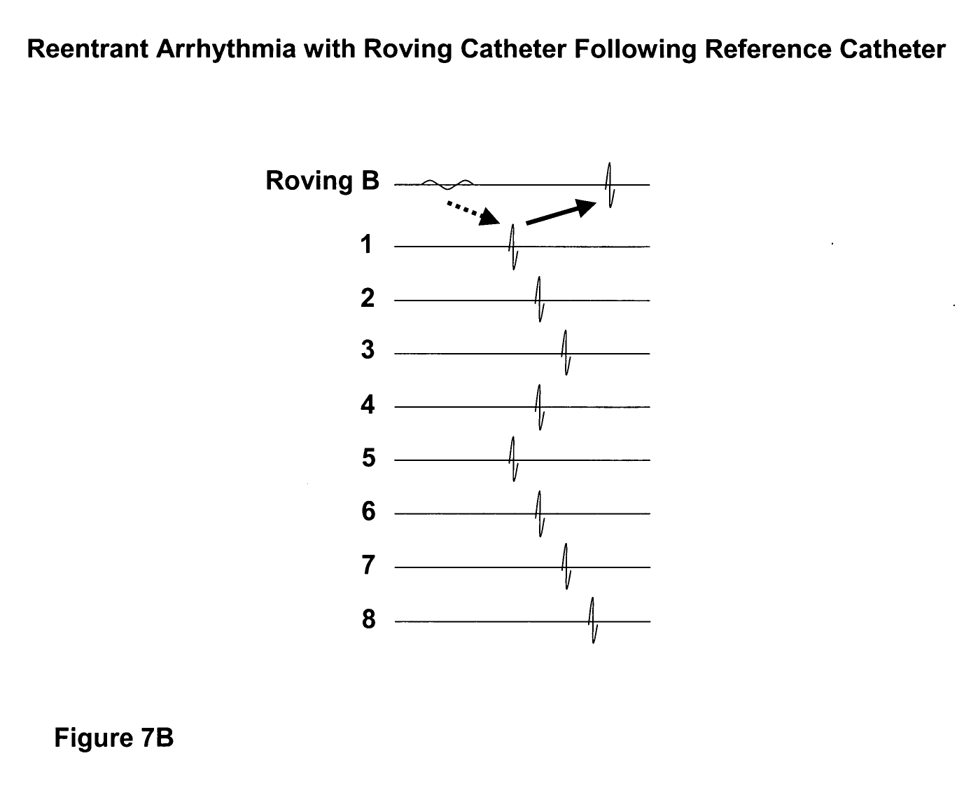

FIG. 7A shows an exemplary illustration of reentrant arrhythmia with the roving catheter in region ‘B’ of FIG. 5 (label 515). Analysis of this electrogram will show that the electrogram feature corresponding to an activation event follows cycle length changes in the reference catheter according to the present invention. Using the averaging technique discussed above, FIG. 7B shows an exemplary illustration with the roving catheter electrogram following cycle length changes in the reference catheter according to the present invention. Alternatively, the pattern-based technique discussed above could also have been used to determine the relationship between roving catheter and reference catheter features.

Information from analysis of FIGS. 5, 6A, 6B, 7A and 7B may be applied to a three-dimensional mapping system whereby regions that are ‘early’ and ‘late’ may be determined. If there is spatial proximity of the ‘early’ and ‘late’ regions, then the arrhythmia may involve a reentrant mechanism with zone of variable conduction time between them. This area may be a region of slow conduction that represents a critical area to the mechanism of the arrhythmia. Application of therapies to this region may result in a cure of the arrhythmia.

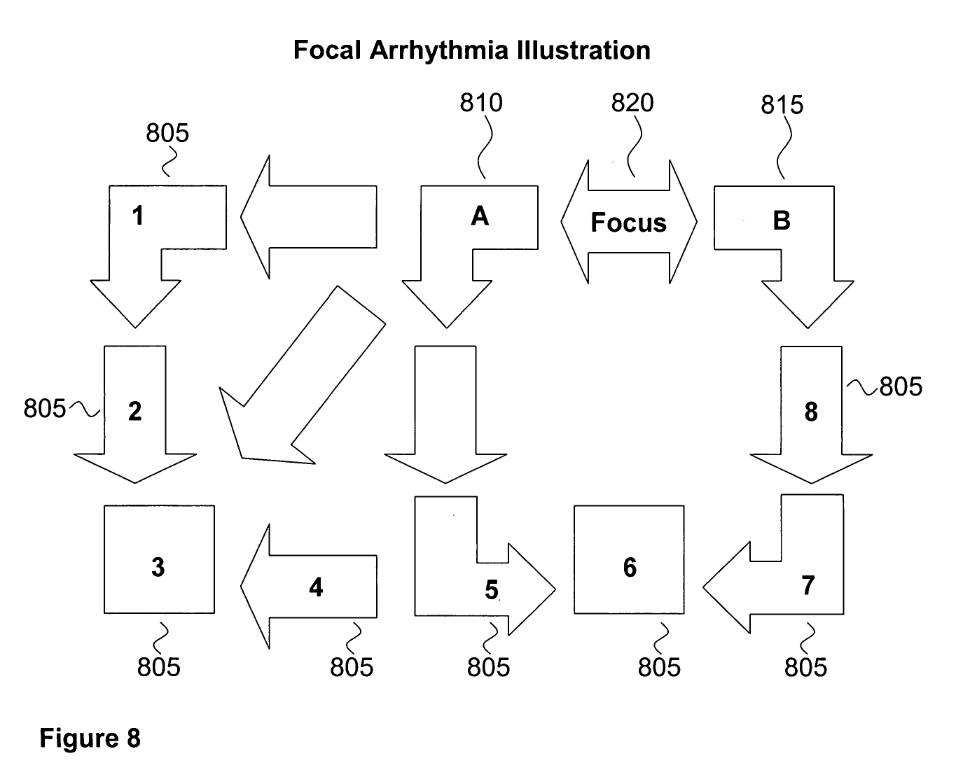

FIG. 8 shows an exemplary illustration of a focal arrhythmia with multiple recording regions according to the present invention. Regions labeled 805 each have a number (1-8) that represents a reference recording electrogram. Similar to FIG. 1, there are regions A and B labeled as 810 and 815, respectively. These regions 810 and 815 may be reached with the roving catheter. In this case, there is also region ‘focus’ labeled as 820. The focus is the origin of a tachycardia and results in a significant portion of the cycle length variation. Identification of the location of the focus and the exit site(s) of the focus may be major targets for further therapies that may result in a cure of the arrhythmia.

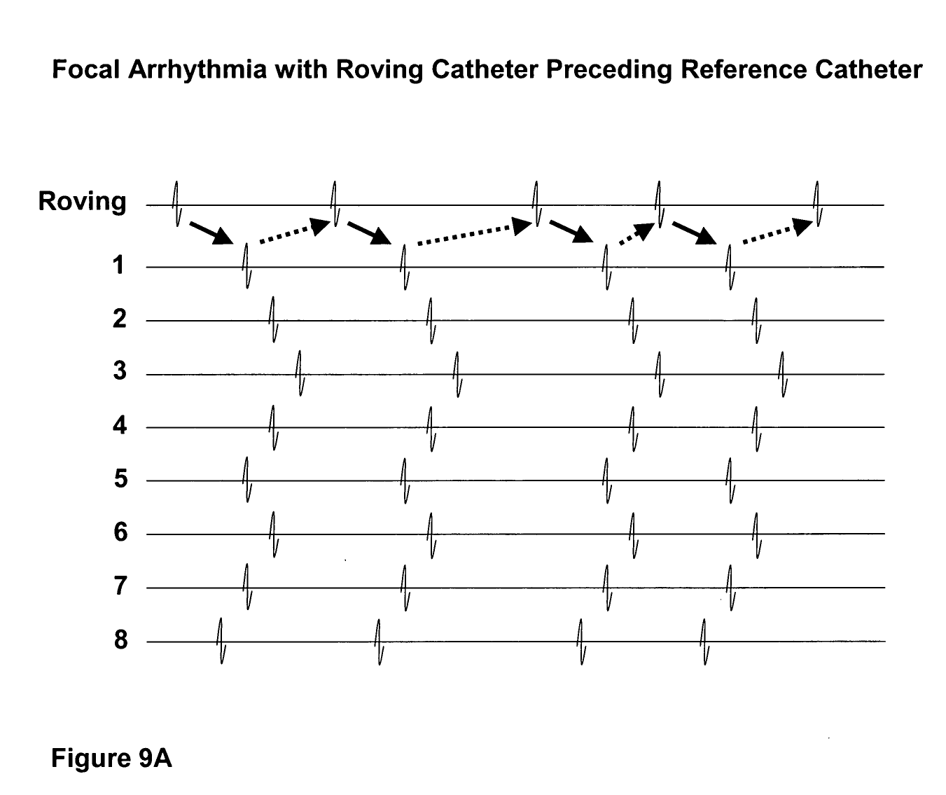

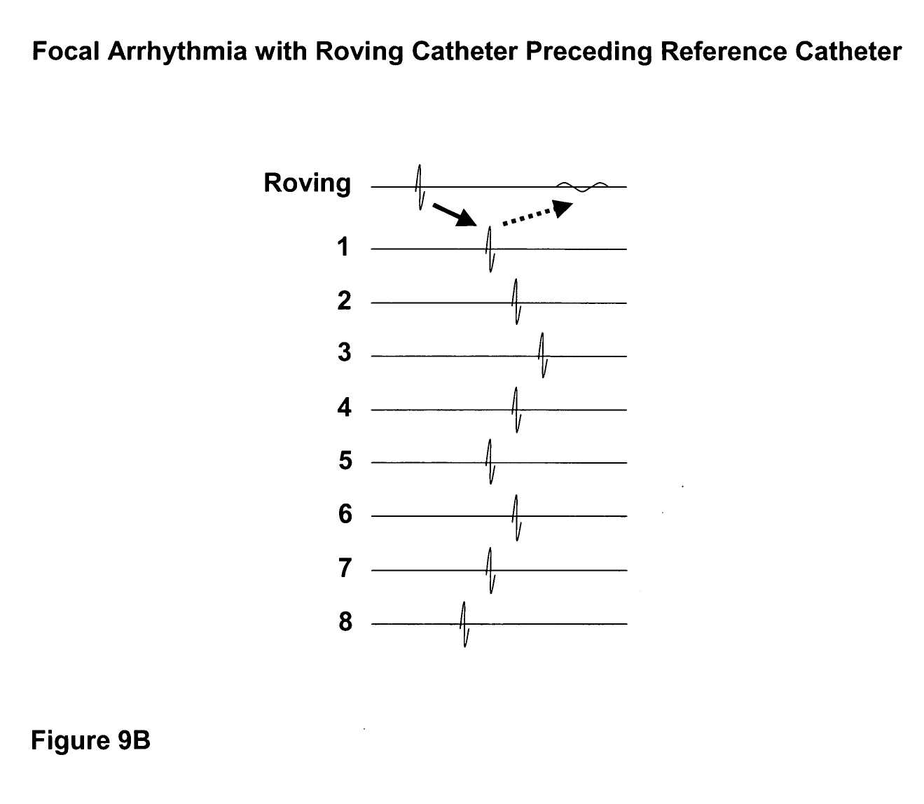

FIG. 9A shows an exemplary illustration of a focal arrhythmia with the roving catheter in region ‘A’ of FIG. 8 (label 810). Analysis of this electrogram will show that the electrogram feature corresponding to an activation event precedes cycle length changes in the reference catheter according to the present invention. Using the averaging technique discussed above, FIG. 9B shows an exemplary illustration with the roving catheter electrogram preceding cycle length changes in the reference catheter according to the present invention. Alternatively, the pattern-based technique discussed above could also have been used to determine the relationship between roving catheter and reference catheter features.

Thus, the exemplary diagnostic method according to the present invention uses timing and variability in timing between electrogram features to determine relationships between those electrogram features. Specifically, the relative timing and variability in conduction times between the electrogram features is determined. The information of timing and variability of conduction times may be incorporated into a three-dimensional mapping system to determine the spatial relationships between the electrogram features. Based upon acquisition of adequate electrograms and analysis of these relationships, the mechanism of an arrhythmia may be determined along with areas that are critical to the mechanism of the arrhythmia.

In the preceding specification, the present invention has been described with reference to specific exemplary embodiments thereof. It will, however, be evident that various modifications and changes may be made thereunto without departing from the broadest spirit and scope of the present invention. The specification and drawings are accordingly to be regarded in an illustrative rather than restrictive sense.

What is claimed is:

1. A method, comprising:
identifying a first plurality of electrogram features in a first electrogram;
identifying a second plurality of electrogram features in a second electrogram; and
determining a relationship between the first plurality of features and the second plurality of features.

2. The method of claim 1, wherein the relationship is a timing and a temporal variability.

3. The method of claim 1, further comprising:
identifying a third plurality of electrogram features in a third electrogram; and
determining a relationship between the third plurality of features and the first and second plurality of features.

4. The method of claim 1, wherein the first electrogram is recorded by one of a reference catheter and the second electrogram is recorded by one of a roving catheter.

5. A method, comprising:
receiving a plurality of electrograms, each electrogram corresponding to a region of a heart, wherein the electrograms are recorded substantially simultaneously;
identifying a plurality of fiducial markers on a selected electrogram, wherein each fiducial marker marks a feature of interest in the first electrogram;
aligning multiple segments of each of the plurality of electrograms, each segment temporally corresponding to each of the fiducial markers on the selected electrogram;
averaging the aligned segments of each of the plurality of electrograms resulting in an average electrogram for each of the plurality of electrograms;
identifying a feature of interest in each of the plurality of average electrograms; and
determining relationships between features of interest in each of the plurality of average electrograms

6. The method of claim 5, wherein the feature of interest is a short duration, high frequency event corresponding to depolarization of the region of the heart near a catheter recording the electrogram.

7. The method of claim 5, wherein the determining of the feature of interest includes one of determining a maximum rate of voltage change, a maximum voltage, a minimum voltage and a maximum correlation to a template.

8. The method of claim 5, wherein the averaging the aligned first plurality of electrograms includes one of direct averaging of voltages, averaging of absolute values of voltages and standard deviation analysis of each point in time along the electrograms.

9. The method of claim 5, wherein the relationships include one of a least variable relationship between the features of interest and a temporal difference between the first and second features of interest.

10. The method of claim 5, further comprising:
mapping the relationships between each electrogram feature of interest in three-dimensional space and onto a three-dimensional model of the heart.

11. The method of claim 5, wherein the selected electrogram is recorded by a reference catheter.

12. The method of claim 5, wherein at least one of the electrograms is recorded by a roving catheter.

13. A method, comprising:
receiving a plurality of electrograms, each electrogram corresponding to a region of a heart, wherein the electrograms are recorded substantially simultaneously;
identifying a plurality of fiducial markers in each electrogram, wherein each fiducial marker marks a feature of interest in the electrogram; and
comparing the fiducial markers in the electrograms to determine relationships between the features of interest.

14. The method of claim 13, wherein the comparing the fiducial markers includes determining temporal differences between a portion of the fiducial markers.

15. The method of claim 13, wherein the portion of the fiducial markers are fiducial markers in the same electrogram.

16. The method of claim 13, wherein the portion of the fiducial markers are fiducial markers in different electrograms.

17. The method of claim 13, wherein the temporal differences indicate variations in conduction time between an electrogram feature of interest corresponding to the portion of the fiducial markers that are compared.

18. The method of claim 13, wherein the features of interest include one of conduction past catheter electrodes in the region and depolarization of the region.

19. The method of claim 13, further comprising:
mapping the relationships between each electrogram feature of interest in three-dimensional space and onto a three-dimensional model of the heart.

20. A signal processing device comprising a processor and a memory storing a set of instructions, wherein the instructions are operable to:
identify a first plurality of electrogram features in a first electrogram;
identify a second plurality of electrogram features in a second electrogram; and
determine a relationship between the first plurality of features and the second plurality of features.

22. A system, comprising:
a plurality of catheters recording a plurality of electrograms, each electrogram corresponding to a region of a heart, wherein the electrograms are recorded substantially simultaneously; and
a signal processing device to receive the plurality of electrograms, identify a plurality of fiducial markers on a selected electrogram, wherein each fiducial marker marks a feature of interest in the first electrogram, align multiple segments of each of the plurality of electrograms, each segment temporally corresponding to each of the fiducial markers on the selected electrogram, average the aligned segments of each of the plurality of electrograms resulting in an average electrogram for each of the plurality of electrograms, identify a feature of interest in each of the plurality of average electrograms and determine relationships between features of interest in each of the plurality of average electrograms.

23. A signal processing device comprising a processor and a memory storing a set of instructions, wherein the instructions are operable to:
receive a plurality of electrograms, each electrogram corresponding to a region of a heart, wherein the electrograms are recorded substantially simultaneously;
identify a plurality of fiducial markers in each electrogram, wherein each fiducial marker marks a feature of interest in the electrogram; and
compare the fiducial markers in the electrograms to determine relationships between the features of interest.
